(19) United States
(12) Patent Application Publication
(10) Pub. No.: US 2012/0150193 A1
(43) Pub. Date: Jun. 14, 2012

(54) SYSTEM AND METHODS FOR HYSTEROSCOPIC TUBULAR LIGATION

(75) Inventors: Lishan Aklog, Scottsdale, AZ (US); Albert K. Chin, Palo Alto, CA (US); Brian deGuzman, Paradise Valley, AZ (US)

(73) Assignee: Pavilion Medical Innovations

(21) Appl. No.: 13/232,801

(22) Filed: Sep. 14, 2011

Related U.S. Application Data
(60) Provisional application No. 61/383,091, filed on Sep. 15, 2010.

(51) Int. Cl. A61B 17/10

(52) U.S. Cl. 606/139

(57) ABSTRACT

A system for tubular ligation is disclosed. The system may include an anchor designed to advance into a channel and engage the walls of the channel. The anchor may have mechanisms that allow it to securely attach to walls of the channel and pull the channel. The system may also include a body for placement adjacent to the channel to align the anchor with the channel. The anchor and body may be arranged so that subsequent retreat of the anchor toward the body pulls and inverts a portion of the channel. The system may also include an occlusion mechanism for placement about the inverted portion of the channel to seal the channel. The occlusion mechanism may be configured to bias between an open state for placement on the body, and a compressed state for sealing the channel. A method for tubular ligation is also disclosed.

### RELATED APPLICATIONS

This application claims priority to and the benefit of U.S. Provisional Application No. 61/383,091, filed Sep. 15, 2010, which is incorporated herein by reference.

### TECHNICAL FIELD

This invention relates generally to tubular ligation, and more particularly, to systems and methods for hysteroscopic tubular ligation of the Fallopian tubes.

### BACKGROUND

Fasteners have been used in surgical procedures to eliminate the need for suturing, which can be both time consuming and inconvenient. In many applications, the surgeon can use an apparatus to deliver the fasteners. Using fasteners may reduce the time required for the procedure and may further reduce complications associated with the procedure, such as blood loss and trauma to the patient.

Various operative procedures previously performed as open surgery requiring relatively large longitudinal incisions have come to be performed endoscopically. In endoscopic procedures, instruments are introduced at internal operative sites through relatively small, artificially created or natural openings providing communication with the internal operative sites from externally thereof. The instruments are manipulated remotely, and/or externally to the operative sites, to perform various operative procedures under visualization provided by an endoscope. Endoscopic procedures have many advantages over open surgical procedures including minimal invasiveness and trauma, shorter hospital stays and recovery times, minimal scarring and patient discomfort, fewer post-operative complications, lower cost and reduced risk for the patient.

Ligating anatomical tissue is a time consuming and tedious part of both endoscopic and open operative procedures due to the difficulty involved in applying an occluding ligature to anatomical tissue as is necessary and desirable in many various procedures. Ligating anatomical tissue is particularly difficult in endoscopic procedures due to the limited room for maneuverability at the internal operative site, the number of different instruments required, and the complicated operative steps involved. In particular, separate instruments are required to attach to the anatomical tissue and to position and contract a ligature loop around the tissue to form a ligature. Furthermore, additional instruments are usually required to cut the ligated tissue as well as the material of the ligature loop. Accordingly, the advantages of endoscopic procedures are sometimes outweighed by the disadvantages caused by the length of time required to perform endoscopic procedures where such time is significantly extended due to the time required for tissue ligation.

The use of endoscopic techniques for tubal ligation has been limited, however, by procedural difficulties due to the limited room for access, maneuverability, and visualization at the operative site. Accordingly, there is a need for a more effective and non-invasive procedure, while minimizing recovery time and risk of injury or infection at the operative site.

### SUMMARY OF THE INVENTION

The present invention provides, in one embodiment, a system for tubular ligation. The system may include a body having a lumen along its length for placement adjacent to an end of a channel. The system may also include an anchor designed for advancement from the lumen and into the channel, in order to engage or attach to walls of the channel, so that subsequent retreat of the anchor toward the body inverts a portion of the channel between the anchor and the body. In one embodiment, the anchor may have mechanisms to securely engage the walls of the channel, such as barbs, flukes, helixes, etc. The system may also include an occlusion mechanism situated on the body for placement about the inverted portion of the channel to seal the channel. The occlusion mechanism may be configured to bias between an activated, open state for placement on the body, and a relaxed, compressed state once off the body for sealing the inverted portion of the channel.

The present invention also provides, in another embodiment, a method for tubular ligation. The method may include placing, adjacent to a channel that is to be inverted, a body having a lumen along its length. An anchor may thereafter be advanced from the lumen and into the channel in order to engage and/or attach to a wall of the channel. Next, the anchor may be retracted towards the body to invert a portion of the channel between the anchor and the body. An occlusion mechanism may then be placed about the inverted portion of the channel in order to seal the channel. The occlusion mechanism may be situated on the outer surface of the body, and may be pushed off the body in order to engage and seal the channel.

### BRIEF DESCRIPTION OF DRAWINGS

FIGS. 1A-1C shows a fastening device in accordance with an embodiment of the present invention.

FIG. 2 shows a fastening device in accordance with an embodiment of the present invention.

FIG. 3 shows a fastening device in accordance with an embodiment of the present invention.

FIG. 4 shows a fastening device in accordance with an embodiment of the present invention.

FIG. 5 shows a fastening device in accordance with an embodiment of the present invention.

FIG. 6 shows a fastening device in accordance with an embodiment of the present invention.

FIGS. 7A-7B shows a fastening device in accordance with another embodiment of the present invention.

FIGS. 8A-8B shows a fastening device in accordance with another embodiment of the present invention.

FIGS. 9A-9B shows a fastening device in accordance with another embodiment of the present invention.

### DESCRIPTION OF SPECIFIC EMBODIMENTS

In accordance with one embodiment of the present invention, systems and methods are provided herein for tubular ligation devices. The devices of the present invention may find use in, for instance, hysteroscopic tubular ligation of the Fallopian tubes. Although discussed here in connection with the Fallopian tubes, it should be appreciated that the device of the present invention can be adapted for ligating other anatomical channels. As used herein, the term “ligate”, “ligating”, or “ligation” may to the act of tying or binding with a ligature, clamp or other device, or otherwise tying or clamping things together and/or occluding a vessel or passage.

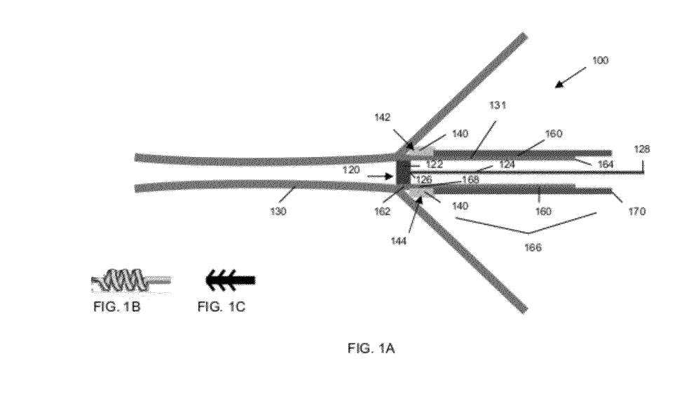

FIG. 1A illustrates a system 100 in accordance with one embodiment of the present invention. The system 100 may provide means of ligating or occluding a vessel, such as a Fallopian tube. In an embodiment, the system 100 of the present invention may include a body 160 having a proximal end 162, a distal end 164, and a lumen 166 extending between the ends. In one embodiment, the body 160 can be designed for placement adjacent to an end of a channel 130, such as, for instance, a Fallopian tube, so as to facilitate occlusion of the channel 130. It should be appreciated, that while described as being placed adjacent a Fallopian Tube, the system 100 of the present invention can be placed adjacent other vessels and/or channels as well.

The body 160, in an embodiment, may be provided with any shape desirable, depending on the particular application, as the shape of the body 160 may affect the ability of the body 160 to be placed adjacent to channel 130. It should be appreciated that while described as a tube, the body 160, of course, may also have any other geometric shape for placement adjacent to an end of channel 130.

In some instances, body 160, may have a diameter sufficient to be placed adjacent to an end of channel 130. In an embodiment, the diameter of the body 160 may remain substantially constant throughout. If desired, the diameter of the body 160 may vary along its length, as necessary. In another embodiment, body 160 may be tapered for placement adjacent to or within an aperture of channel 130.

The body 160, in another embodiment, may have a length sufficient to permit the body 160 to be inserted into and advanced through a paitent's body to a site adjacent to channel 130.

In one embodiment, the body 160 can be made from any material capable of passing through the body of a patient and to a site adjacent to channel 130. To that end, body 160 may be formed from a substantially hard material so as to minimize circumferential deformation of the body 160 during delivery. Examples of materials that are substantially hard include metals, plastics, ceramics, composite material, or combination thereof, as well as any other materials that can maintain a substantially consistent shape.

In certain situations, the body 160 can be made from a substantially flexible or malleable material so as to allow bending or deformation of the body 160 during advancement to a site of interest and/or during use of the system. Examples of materials that are substantially flexible or malleable include metals, plastics, ceramics, shape memory material, or any other materials that can allow deformation of its shape.

Since body 160 is designed to be inserted into the body of a human or animal, body 160, in an embodiment, can be made from a material that is biocompatible. The biocompatibility of the material may help minimize occurrence of adverse reactions due to use of the body 160 within the body of the patient. Body 160 may further include a coating on an outer surface to reduce friction between body 160 and the patient's body. Likewise, the body 160 may include a coating on an inner surface to reduce friction during deployment of the device 110 situated within the body 160.

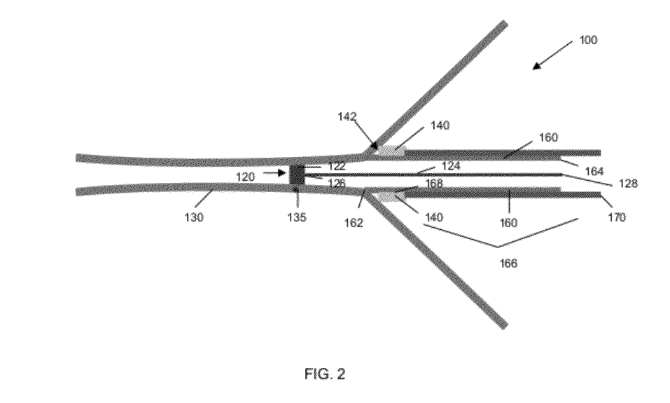

As shown in FIG. 1A, the system 100 may also include, in an embodiment, a inversion mechanism 120 designed for advancement along the lumen 166 distally beyond the body 160. As shown in FIG. 2, the inversion mechanism 120 may be designed to advance distally beyond an end of the body 160 and along a channel 130, such as, a Fallopian Tube. As shown, body 160 may act to align inversion mechanism 120 with channel 130 to facilitate advancement of inversion mechanism 120 into channel 130. As will be discussed below, the inversion mechanism may provide means for inverting channel 130 so as to expose the inner surface of channel 130, in order to facilitate occlusion and/or ligation of channel 130.

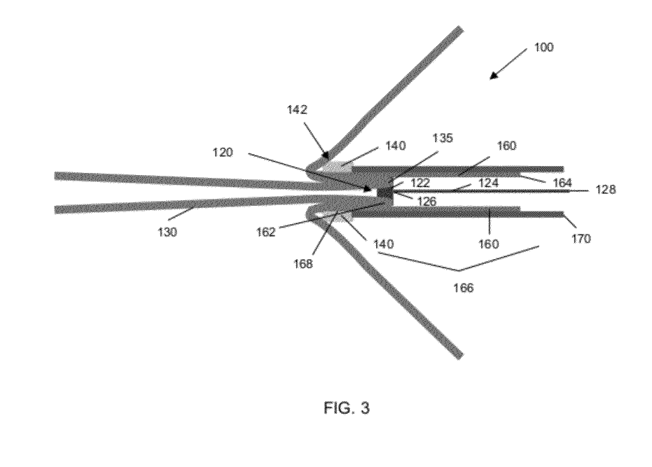

The inversion mechanism 120, in one embodiment, may include an anchor 122 for engaging the walls of channel 130. In some embodiments, anchor 122 may advance into channel 130 and attach to channel 130 at a desired point on the inner wall of channel 130, so that subsequent retreat of the anchor 122 proximally towards the body 160 can pull upon channel 130. Pulling upon channel 130 in this way may act to invert a portion of channel 130 between anchor 122 and body 160. In some instances, as anchor 122 retreats toward body 160, anchor 122 may draw a portion of channel 130 into body 160 so that channel 130 becomes inverted, as shown in FIG. 3.

The point at which the anchor 122 attaches to the inner wall of the channel 130 may be referred to as the “inversion point” 135, or the point at which the channel 130 inverts upon itself, as shown in FIG. 3. The inversion point 135, in an embodiment, may be a point situated at a sufficient distance along the channel 130 to allow the inversion point 135 to be engaged and pulled toward the body 160 to a site, such as the uterine cavity, where the channel 130 can subsequently be ligated.

The anchor 122, in accordance with the present invention, can be provided with a variety of shapes and designs. As shown in FIG. 1A, for instance, the anchor 122 can be provided with a rounded design. If desired, however, the anchor 122 may be provided with other designs, such as, for instance, a helix or double-helix, as shown in FIG. 1B, or barbs and/or flukes, as shown in FIG. 1C. Of course, other designs are also possible so long as they permit the anchor 122 to advance along the channel 130 and attach to the inner wall of channel 130.

It should be appreciated that the anchor 122 can have any diameter desired so long as the diameter permits the anchor 122 to travel within the body 160, and further allows the anchor 122 to be accommodated within the channel 130. In addition, it should be appreciated that anchor 122 should be provided with a length that permits it to be advanced a sufficient distance within the channel 130 to allow it to sufficiently engage the wall of channel 130. In some embodiments, the diameter of the anchor may be adjustable so that the anchor 122 can advance into channel 130 and engage the inner surface of channel 130. For instance, anchor 122 may have a relatively small diameter or a slim profile for advancing through body 160 and into channel 130. Once within channel 130, the diameter of anchor 122 may be increased so as to engage and attach to the inner surface of channel 130 so that channel 130 may be pulled into body 160 for inversion. Of course, if desired, the inversion mechanism 120 may have other dimensions as well.

To adequately engage the anchor 122 to a site along the length of the channel 130, the anchor 122 can be made from a material and provided with a design that can latch onto and attach to channel 130. In an embodiment, the anchor 122 can be made from a material that is relatively strong to maintain the channel 130. In another embodiment, the anchor 122 may be formed from a shape memory, biasing, or spring material.

In one embodiment, the anchor 122 can also be made from a material that allows for its subsequent elimination once the attaching or engaging function is no longer necessary. As used herein, the term “elimination” can be understood to mean manual removal of the element or otherwise. In one embodiment, the anchor 122 can be made from a material that is capable of being severed or broken. Such a material would allow for manual removal of the anchor 122. In another embodiment, a device can be used to remove the anchor 122. In other embodiments, the anchor 122 can be made entirely or partially from material that is bioresorbable or biodegradable. In such instances, the anchor 122 may be entirely or partially absorbed by the body after a certain period of time had elapsed and would eliminate the need for manual removal of the anchor 122.

In an embodiment, the material from which the anchor 122 may be formed includes metal, metal alloy, polymer, molded plastic, metal-polymer blend, or a combination thereof. The type of material may affect the strength and/or flexibility of the anchor 122. Examples of suitable materials include shape memory material, stainless spring steel, superelastic metal such as Nitinol, rigid plastic such as polycarbonate, Ultem, or LCP (liquid crystal plastic), or rigid absorbable compounds such as PGA (polyglycolic acid). Other suitable materials include gold, platinum, tungsten, nickel-titanium alloy, Beta III Titanium, cobalt-chrome alloy, cobalt-chromium-nickel-molybdenum-iron alloy, Elgiloy, L605, MP35N, Ta-10W, 17-4PH, Aeromet 100, polyethylene terapthalate (PET), polytetraflouroethylene (PTFE), polyurethane (nylon) fluorinated ethylene propylene (FEP), polyurethane, polypropylene (PP), polyvinylchloride (PVC), polyether-ester, polyester, polyamide, elastomeric polyamides, block polyamide/ethers, polyether block amide (PEBA), silicones, polyethylene, polyether-ether ketone (PEEK), polyimide (PI), polyetherimide (PEI), tantalum, tungsten, or any other suitable material that is biocompatible. The inversion mechanism 120 may also include an anti-thrombogenic coating such as heparin (or its derivatives), urokinase, or PPack (dextrophenylalanine proline arginine chloromethylketone) to prevent thrombosis or any other adverse reaction from occurring at the site of insertion.

Additionally, since the anchor 122 is designed to be implanted within the body of a human or animal, the anchor 122 should be made from a material that is biocompatible. The biocompatibility of the material may help minimize occurrence of adverse reactions due to implantation of the inversion mechanism 120 within the body.

The inversion mechanism 120, in an embodiment, can also include an elongated body 124 designed to advance the anchor 122 to a site within the channel 130 (e.g. a site within the Fallopian tubes). The elongated body 124, in an embodiment, may have a proximal end 126 and a distal end 128. At its distal end 128, the elongated body 124 may be coupled to the anchor 122. In one embodiment, the elongated body 124 may be detachably coupled to the anchor 122 to allow for subsequent detachment from the anchor mechanism 122 once the anchor mechanism has been used to draw channel 130 into body 160, for example. In certain circumstances, if the anchor is attached to channel 130, detachment of the anchor may be desirable so that the system 100 may be removed from the patient without pulling upon and damaging channel 130.

It should be appreciated that the elongated body 124 may be provided with any length or shape desirable, depending on the particular application, as the shape of the elongated body 124 may affect ability of the elongated body 124 to deliver the anchor 122 to a site for delivery. For instance, elongated body 124 may be tubular in shape. It should be appreciated that while described as a tube, the elongated body 124, of course, may have any other geometric shape as well.

It should further be appreciated that the elongated body 124 may have any diameter or size desired so long as the diameter or size permits it to be accommodated within body 160.

In one embodiment, the elongated body 124 can be made from any material capable of passing through the body 160 and delivering the anchor 122 to a site of delivery. To that end, elongated body 124 may be formed from a substantially hard material so as to minimize deformation of the elongated body 124 during delivery. Examples of materials that may be used include metals, plastics, ceramics, or any other materials that can maintain a substantially consistent shape.

In certain situations, the elongated body 124 can be made from a substantially flexible or malleable material so as to allow bending or deformation of the elongated body 124 during delivery. Examples of materials that may be used include metals, plastics, ceramics, or any other materials that can allow deformation of its shape.

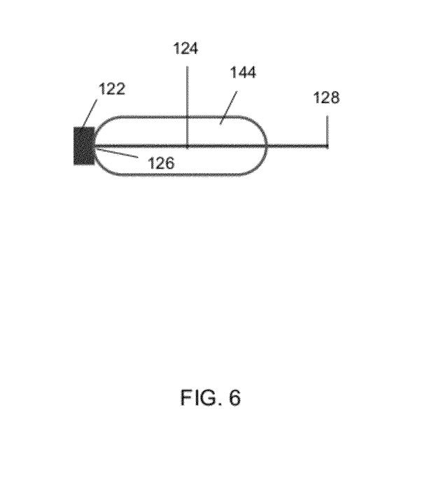

In order to aid in the retreat of the anchor 122 during inversion of the channel 130, the system 100 of the present invention may be provided with a balloon 180, as shown in FIG. 6. The balloon 180 may be designed to aid in the dilation or expansion of the channel 130 during inversion. For example, balloon 180 may have a diameter that dilates or expands channel 130 beyond that of the anchor 122 so that during retreat of anchor 122 towards body 160, obstructions by or within channel 130 can be minimized. Balloon 180 may act to provide structure or support to channel 130 so that, as anchor 122 is drawn toward body 160, balloon 180 can minimize the occurrence of channel 130 becoming folded, creased, or otherwise crumpled during inversion. One skilled in the art will recognize that a balloon similar to balloon 180 can also be used to minimize obstruction while anchor 122 is advanced away from body 160 and into channel 130.

In some embodiments, balloon 180 may be inflated and deflated in order to dilate channel 130. While inflated, balloon 180 may press or touch against the walls of channel 130 in order to support the walls. Balloon 180 may also be deflated so that it can be accommodated within body 160 and advanced into channel 130. In an embodiment, balloon 180 may be situated in a position proximal and adjacent to anchor 122 and about the elongated body 170, as shown in FIG. 6.

Once channel 130 is inverted, it may be desirable to occlude or close channel 130. Accordingly, the system 100 may further include an occlusion mechanism 140. Occlusion mechanism 140 may be designed for placement about the inverted channel 130 to seal the channel 130 and minimize access or movement across the point of inversion 135. In other words, the occlusion mechanism 140 may act as plug or ligature to close the channel 130 to prevent movement of fluids or other biological products through the channel 130. By forming a ligature or a seal about the channel 130, the occlusion mechanism 140 may reduce the need for making surgical incisions, and thus may reduce the likelihood of pain or wound infection.

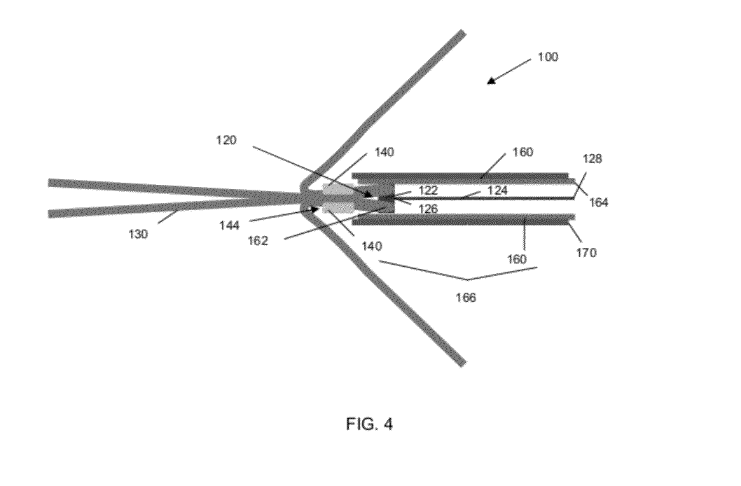

In some embodiments, occlusion mechanism 140 may situated on an outer surface 168 of the body 160, as shown in FIG. 3, near the point of inversion. Locating occlusion mechanism 140 on the outer surface of body 160 may allow occlusion mechanism 140 to engage an inverted surface of channel 130 in order to plug or close channel 130, as shown in FIG. 4. Occlusion mechanism 140 may be located anywhere along the length of body 160. In some instances, occlusion mechanism 140 may be located at a distal end of body 160 so that occlusion mechanism 140 may engage the inverted surface of channel 130.

In one embodiment, the occlusion mechanism 140 may be capable of biasing between an actuated state and a resting state. In the actuated position 142, the occlusion mechanism 140 may be expanded as it is situated on the outer surface 168 of the body 160, as shown in FIG. 3.

Occlusion mechanism may also be biased into a resting position 144 (shown in FIG. 4) where the diameter of the occlusion mechanism 140 is compressed so that it may ligate, plug, or otherwise seal channel 130. For example, following inversion of the channel 130 by the inversion mechanism 120, the occlusion mechanism 140 may be designed to be pushed off of body 160 and revert to its resting or compressed position 144 about channel 130. In the resting position 144, the occlusion mechanism 140 can be compressed about the inverted channel 130 to seal the channel 130 and minimize access or movement across the point of inversion 135. In this compressed position, occlusion mechanism 140 may be situated about channel 130 so as to compress channel 130 between portions of occlusion mechanism 140.

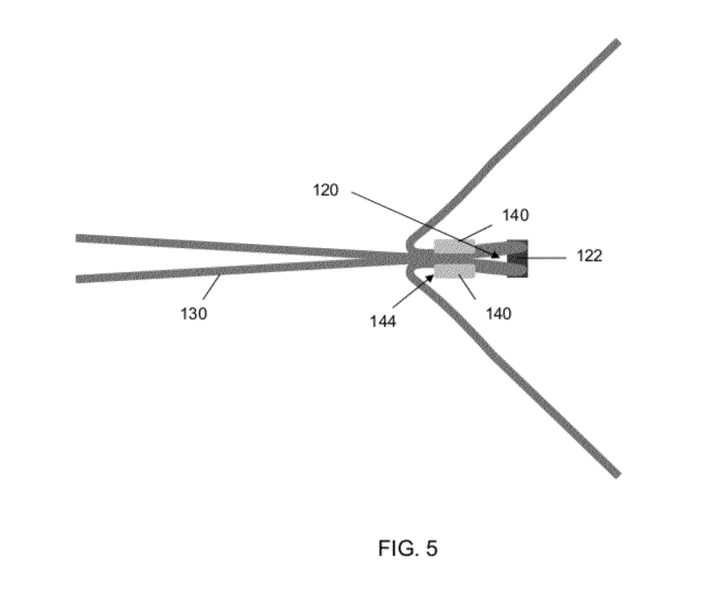

The occlusion mechanism 140 may be provided with a variety of designs. In one embodiment, the occlusion mechanism 140 may be a band that acts to seal the pathway 130 from all sides. For example, occlusion mechanism 140 may be a ligature, elastic, suture, etc., that can be compressed about channel 130 so as to close channel 130. In an alternative embodiment, the occlusion mechanism 140 may be designed to secure the channel 130 from two sides, as shown in FIGS. 4-5. For example, the occlusion mechanism 140 may be a clamp, press, clip, or crimp that can close channel 130 by compressing together two (or more) opposing sides of channel 130. It should be noted that while shown to secure the channel 130 from all sides or from opposing sides, the occlusion mechanism 140 may be designed to secure the channel 130 in other ways as well.

To secure the channel 130 from more than one side, the occlusion mechanism 140 may include more than one component. As shown in FIG. 1A, the occlusion mechanism 140 may be provided with two components 142 and 144. The two components 142 and 144 may compress toward one another to allow for closure of the channel 130 from two opposing sides. While described herein as being provided with two components, it should be appreciated that the present invention may be provided with more than two or less than two components that can seal channel 130 as well.

To enhance closure of the channel 130 and minimize access or movement across the point of inversion 135, the components 142 and 144 may be provided with a complimentary design. In some instances, as shown in FIG. 1A, components 142 and 144 may have rectangular designs so they can be pressed toward one another to seal channel 130. Of course, the occlusion mechanism 140 can also be provided with a variety of other geometrical shapes and designs so long as they permit the occlusion mechanism 140 to adequately secure the channel 130.

To adequately secure the channel 130, the occlusion mechanism 140 should be made from a material that is relatively strong. Additionally, since the occlusion mechanism 140 is designed to be implanted within the body of a human or animal, the occlusion mechanism 140 should be made from a material that is biocompatible. The biocompatibility of the material may help minimize occurrence of adverse reactions due to implantation of the occlusion mechanism 140 within the body. Examples of materials from which the occlusion mechanism 140 may be formed includes metal, metal alloy, polymer, molded plastic, metal-polymer blend, or a combination thereof, all of which are described in greater detail above.

In certain embodiments, the system 100 of the present invention may be designed to allow a guide (not shown) to help direct the system 100 through the body. The guide may be designed to be positioned in such a manner to maintain the stability of the system 100 as the system 100 is advanced along the body. It should be noted that while the guide can be positioned in any manner to allow guidance of the system 100, its design should minimize any obstructions of the system 100. In an embodiment, the guide may be any guide that is commercially available.

In an embodiment, the system 100 may be provided with a outer sheath 170. The outer sheath 170 may be designed to advance the occlusion mechanism 140 from the body 160, as shown in FIG. 3, onto the inverted channel 130, as shown in FIG. 4. In some embodiments, the outer sheath 170 may be slidably engaged on an outer surface of body 160, as shown in FIG. 3, so that the outer sheath 170 may slide along the length of the body 160 in order to push and advance occlusion mechanism 140 along the length of the body 160. Of course, it should be appreciated that the outer sheath 170 may be situated in any other manner as long as the outer sheath 170 is capable of pushing the occlusion mechanism 140 off the body 160.

It should also be appreciated that the outer sheath 170 may be provided with any shape desirable, depending on the particular application, as the shape of the outer sheath 170 may affect the ability of the outer sheath 170 to advance the occlusion mechanism 140 from the body 160. For instance, outer sheath 170 may be tubular in shape. It should be appreciated that while described as a tube, the outer sheath 170, of course, may have any other geometric shape as well.

The outer sheath 170, in another embodiment, may have a diameter sufficient to allow the body 160 to be placed therein. In an embodiment, the diameter of the outer sheath 170 may remain substantially constant throughout. If desired, the diameter of the outer sheath 170 may vary, as necessary. In addition, the outer sheath 170, in another embodiment, may have any length desired so long as the length is sufficient to accommodate the length of the body 160. One skilled in the art will recognize that, although depicted as an outer sheath that surrounds body 160, outer sheath 170 need not surround body 160 and may have any design so long as outer sheath 170 can push occlusion mechanism 140 off of body 160 to seal channel 130.

In one embodiment, the outer sheath 170 can be made from any material sufficiently straight to permit the outer sheath 170 to be directed through the body of a patient to a site adjacent a channel 130. To that end, outer sheath 170 may be formed from a substantially hard material so as to minimize deformation of the outer sheath 170 during delivery. Examples of materials that are substantially hard include metals, plastics, ceramics, or any other materials that can maintain a substantially consistent shape.

In certain situations, the outer sheath 170 can be made from a substantially flexible or malleable material so as to allow bending or deformation of the outer sheath 170 during delivery. Examples of materials that are substantially flexible or malleable include metals, plastics, ceramics, or any other materials that can allow deformation of its shape.

Since the outer sheath 170 is designed to be inserted into the body of a human or animal, the outer sheath 170, in an embodiment, can be made from a material that is biocompatible. The biocompatibility of the material may help minimize occurrence of adverse reactions due to use of the outer sheath 170 within the body. The outer sheath 170 may further include a coating on an outer surface to reduce friction between the outer sheath 170 and the body upon insertion into the body. Likewise, the outer sheath 170 may include a coating on an inner surface to reduce friction during any movement of the body 160.

The system 100, in accordance with an embodiment, may also be provided with a deploying mechanism (not shown) from which sufficient force can be applied to advance the inversion mechanism 120 to a site within the channel 130. In one embodiment, the deploying mechanism can be any mechanical design sufficient to advance and/or retract the inversion mechanism 120. Of course, other designs of the deploying mechanism, such as a hydraulic mechanism that applies liquid or fluid pressure to inversion mechanism 120, may also be used to advance and/or retract inversion mechanism 120. The deploying mechanism may act upon inversion mechanism 120, or any portion of inversion mechanism 120, including, but not limited to anchor 122, elongate body 124, or any other portion of inversion mechanism 120. Other designs are also within the scope of the invention, as the present invention is not intended to be limited in this manner.

To prepare the system 100 for insertion in the body, a user can initially insert a guide to a site adjacent a channel 130. Once inserted, the guide may be used to help direct the body 160 to the site adjacent a channel 130. After the body 160 is advanced to the site, an inversion mechanism 120 may be positioned within the body 160. The anchor 122 of the inversion mechanism may be situated adjacent the distal end 164 of the body 160 and the proximal end 128 of the elongated body 124 may be situated adjacent the proximal end 162 of the body 160. Additionally, occlusion mechanism 140 can be inserted and positioned on the outer surface 168 of the body 160 adjacent the distal end 164. A outer sheath 170 may also be situated about the body 160 in such a manner to allow the outer sheath 170 to push the occlusion mechanism 140 from the body 160 onto the inverted channel 130.

Once at the site adjacent to the channel 130, the system 100 can be prepared for delivery. The body 160 may be positioned so as to align the inversion mechanism 120 and/or anchor 122 with channel 130. Delivery may then include advancement of the inversion mechanism 120 along the lumen 166 of the body 160 distally beyond the body 160. As the inversion mechanism 120 is advanced, the anchor 122 may be advanced into channel 130 until it reaches a desired point of inversion 135, at which point the anchor 122 engages the walls of the channel 130. After the anchor 122 is anchored to the channel 130, the inversion mechanism 120 may be subsequently retracted proximally towards the body 160. This withdrawal may act to pull a portion of channel 130 into body 160 so that channel 130 becomes inverted, as shown in FIG. 3.

After the channel 130 is inverted, the outer sheath 170 may push the occlusion mechanism 140 from the body 160 onto the inverted portion of channel 130 to subsequently seal channel 130, as shown in FIG. 4. With the occlusion mechanism 140 secured in place about the channel 130, channel 130 may be blocked and movement across the point of inversion 135 is minimized. Once the occlusion mechanism 140 is in place, the elongated body 124 may be detached from the anchor 122 and removed from the body along with the body 160, as shown in FIG. 5.

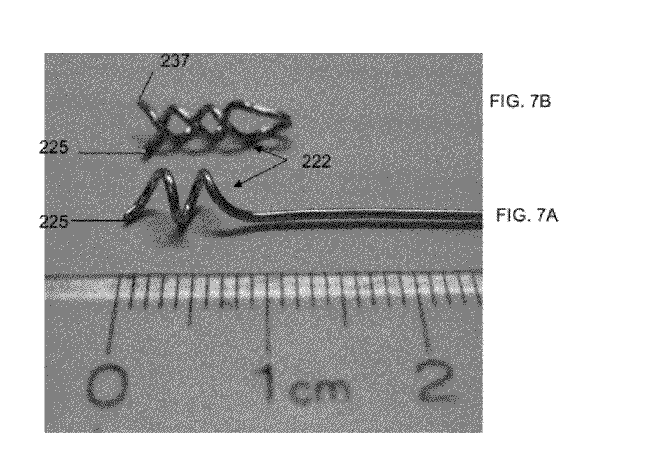

Alternate embodiments for tubal ligation are illustrated in FIGS. 7A-9B. Referring now to FIG. 7A, anchor 222 may be provided with a corkscrew, or helical coil. Upon rotation of the anchor 222, the tip 225 of the coil may penetrate the wall of channel 130 in a helical pattern in order to secure itself into the wall of the channel 130. As such, tip 225 may be sufficiently sharp and strong to anchor itself into the channel 230. Rotation may be either clockwise or counterclockwise, depending on the application.

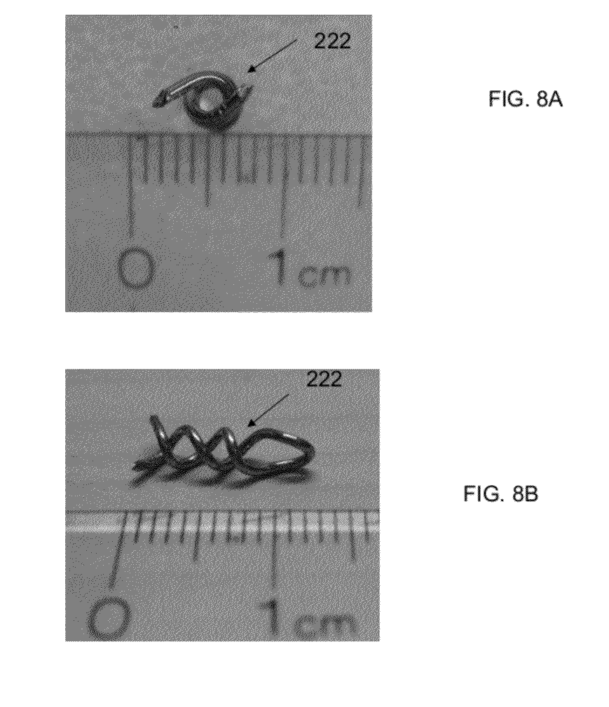

In some embodiments, anchor 222 may be provided with opposing corkscrews that are combined, as shown in FIG. 7B. In this arrangement, the coils may be arranged in a double-helix pattern. FIGS. 8A-8B show the axial and side views, respectively, of this double-helix arrangement. It should be appreciated that while described as a single coil or double-helix arrangement, anchor 222 can be formed from more than two coils as well.

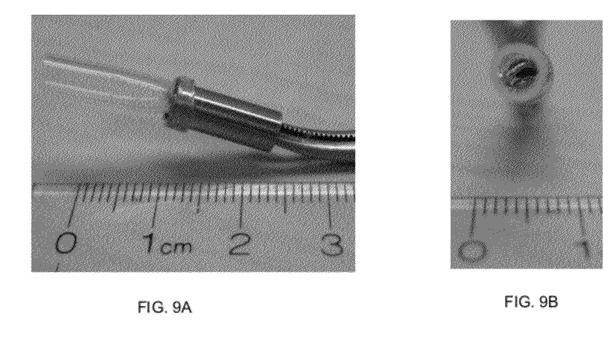

Similar to the embodiment described above, upon rotation of the double-spiral anchor 222, the tips 225 of the spiral coil may be designed to anchor or secure themselves into the wall of the channel 130. In such a configuration, the anchor may be designed so that, as it is rotated into the wall of channel 130, it compresses the wall of channel 130 between adjacent coils. Compressing the walls of channel 130 in this way may decrease the caliber of the channel 130 at the point where anchor 222 engages channel 130, which may facilitate inverting channel 130 at the point of engagement. The body 160, in an embodiment, may act to expand the channel 130 at its proximal end (i.e., the end where the channel 130 and the body 160 are adjacent one another), thereby enhancing traction at that proximal end. Added traction may, in one embodiment, allow the double-helix anchor 22 to intussuscept or invaginate the channel 130, or otherwise turn at least a portion of channel 130 “inside-out,” as shown in FIGS. 9A-9B.

It should be appreciated that while described herein as finding use in medical applications, the system 100 of the present invention may also find use in non-medical applications where ligating or tying channels may be necessary.

While the invention has been described in connection with the specific embodiments thereof, it will be understood that it is capable of further modification. Furthermore, this application is intended to cover any variations, uses, or adaptations of the invention, including such departures from the present disclosure as come within known or customary practice in the art to which the invention pertains, and as fall within the scope of the appended claims.

What is claimed is:

1. A system for tubular ligation, comprising:
an anchor designed for advancement into a channel to engage walls of the channel;
a body, defining a lumen along its length, for placement adjacent to an end of the channel to align the anchor with the channel; and
a retraction mechanism extending from the lumen and coupled to the anchor, such that retreat of the retraction mechanism can pull the anchor toward the body and invert a portion of the channel between the anchor and the body.

2. A system as set forth in claim 1, wherein the anchor includes a mechanism to securely engage the walls of the channel to secure the anchor within the channel.

3. A system as set forth in claim 2, wherein the anchor includes one or more barbs, flukes, helixes, or designs to secure itself within and pull the channel.

4. A system as set forth in claim 1, wherein the anchor is designed to disengage from the retraction mechanism to allow the anchor to remain in the channel.

5. A system as set forth in claim 1, wherein the anchor is constructed from a bioadsorbable material.

6. A system as set forth in claim 1, wherein the body is sufficiently flexible to allow bending of the body.

7. A system as set forth in claim 1, wherein the retraction mechanism is an elongate body extending through the lumen for advancing the anchor into the channel and retracting the anchor toward the body.

8. A system as set forth in claim 1, further comprising an occlusion mechanism, situated on the body, for placement about the inverted portion of the channel to seal the channel.

9. A system as set forth in claim 8, wherein the occlusion mechanism can bias between an activated, open state for placement on the body, and a relaxed, compressed state once off the body for sealing the channel.

10. A system as set forth in claim 9, wherein the occlusion mechanism is one of a ligature, a clamp, a suture, or a combination thereof.

11. A system as set forth in claim 1, further comprising a sheath, slidably engaged about an outer surface of the body, for advancing an occlusion mechanism off the body to engage the inverted portion of the channel.

12. A system as set forth in claim 1, further comprising a balloon situated on a proximal side of the anchor to dilate the channel in order to minimize obstruction during retraction of the anchor and inversion of the channel.

13. A system as set forth in claim 1, wherein the body, the anchor, the retraction mechanism, or a combination thereof are constructed from a biocompatible material.

14. A method for tubular ligation, comprising:
placing a body adjacent to a channel so as to align an anchor with the channel, the body defining a lumen along its length;
advancing the anchor into the channel in order to engage a wall of the channel; and
retracting the anchor towards the body to invert a portion of the channel between the anchor and the body.

15. A method as set forth in claim 14, wherein the step of placing includes arranging the body so that the body is in axial alignment with the channel.

16. A method as set forth in claim 14, wherein the step of advancing includes attaching the anchor to the walls of the channel with an attachment feature of the anchor.

17. A method as set forth in claim 14, wherein the step of retracting includes dilating the channel with a balloon as the anchor retreats so as to minimize obstruction as the channel is inverted.

18. A method as set forth in claim 14, further comprising positioning an occlusion mechanism about the inverted portion of the channel in order to seal the channel.

19. A method as set forth in claim 18, wherein the step of positioning includes allowing the occlusion mechanism to bias to a compressed state once it is off the body for sealing the channel.

20. A method as set forth in claim 18, wherein the step of positioning includes pushing the occlusion mechanism off the body onto the inverted portion of the channel in order to seal the channel.

21. A method as set forth in claim 20, wherein the step of pushing includes moving a sheath along the body in order to push the occlusion mechanism off the body and onto the inverted portion of the channel.

22. A method as set forth in claim 14, further comprising decoupling, from the anchor, an elongate member for advancing and retracting the anchor, so that the anchor may remain within the channel upon removal of the elongate member from the site of ligation.
